(19) United States
(12) Patent Application Publication
(10) Pub. No.: US 2008/0091198 A1
(43) Pub. Date: Apr. 17, 2008

(54) Bone Fusion Plate

(75) Inventors: David A. Leibel, Princeton, MN (US); William P. Cooney III, Rochester, MN (US); Ronald Linscheid, Rochester, MN (US); Richard Berger, Rochester, MN (US)

Correspondence Address: MORGAN, LEWIS & BOCKIUS LLP, 1701 MARKET STREET, PHILADELPHIA, PA, 19103-2921, US

(73) Assignee: MAYO Foundation for Medical Research and Education, Rochester, MN (US)

(21) Appl. No.: 11/951,730

(22) Filed: Dec. 6, 2007

Related U.S. Application Data
(62) Division of application No. 10/909,259, filed on Jul. 30, 2004.

(51) Int. Cl. A61B 17/58; A61B 17/00; A61B 17/56

(52) U.S. Cl. 606/60; 606/73; 606/85

(57) ABSTRACT

A carpal fusion plate for use in fusion of the bones of the midcarpus or other bones. The fusion plate is concave and defines at least one round countersunk screw hole and at least one tapered elongate slotted aperture. The tapered elongate slotted aperture is narrower peripherally and broader centrally. The fusion plate also defines a multilobated central opening.

### FIELD OF THE INVENTION

The invention relates to internal fixation devices utilized in orthopedic surgery. More particularly, the invention relates to an internal fixation device to be implanted within the body in direct contact with a bone or bone fragment to reinforce the bone or bone fragment during the healing process or for positioning a plurality of bones or bone fragments relative to one another.

### BACKGROUND

Orthopedic fusion of damaged or degenerated bones in a joint is well known in the orthopedic arts. Joint fusion involves the fixing of several bones relative to one another so that they are no longer moveable relative to one another and so that they subsequently move as a unit. Typically joint fusion is accomplished by securing an appliance to the bones that are to be fused. Very often joint fusion involves the packing of bone graft material between the fixed bones to encourage the bones to grow together. A bone graft consists of fragments of bone removed from another location in the patient's body. The living bone material will ossify in place and bridge the joint space and cause the adjacent bone to fuse. Joint fusion may be performed at many locations in the body.

This application will discuss four-corner fusion of the lunate, capitate, hamate and triquetrum bones of the carpus in some detail. It is to be understood that the invention disclosed herein may also be utilized to assist in the fusion of other bone including but not limited to fusion of the tarsal-metatarsal complex in the foot.

When practiced on the human wrist, wrist fusion can vary in extent. Wrist fusion can be virtually complete involving fusion of the radius, most or all of the carpal bones, and at least one of the metacarpals. This type of fusion results in immobilization of virtually the entire wrist joint. While this approach to fusion of the wrist may be necessary in some cases to relieve chronic and severe pain from certain forms of arthritis, it is preferable, if possible, to maintain as much natural motion of the wrist joint as possible.

Limited wrist fusion is often required in the treatment of advanced arthritis of the wrist. Two common conditions seen in advanced arthritis are scapholunate dissociation advanced collapse (SLAC wrist) and scaphoid non-union arthritic advanced collapse (SNAC wrist). Limited wrist fusion may also be required because of failed treatment of perilunate fracture-dislocations of the wrist.

In limited wrist fusion, several of the bones of the carpus are fused together without fusing the metacarpals to the carpals or the radius to the carpals. A common limited wrist fusion procedure is known as “four corner” fusion. Four corner fusion involves fusing the capitate, hamate, lunate, and triquetrum bones in the carpus. An appliance for use in four corner limited wrist fusion is disclosed in U.S. Pat. No. 6,179,839 B1, issued to Weiss and Collins. The appliance disclosed in the '839 patent can be used for four corner fusion, as well as for other fusion procedures. The '839 appliance is an annular conical plate having a top outer edge of greater diameter and a smaller diameter inner bottom edge. The appliance is substantially conical in shape based upon a cone having an angle of roughly 35° with the base.

When the device disclosed in the '839 patent is implanted for four corner fusion a conical burr is used to rasp out a conical cavity at the juncture of the capitate, hamate, lunate, and triquetrum bones in the human carpus. The appliance is then placed in the resulting conical cavity and bone fasteners, such as bone screws, are inserted through holes in the appliance into the capitate, hamate, lunate, and triquetrum bones to pull the bones together. Typically, two screws are inserted into each carpal bone.

As discussed above, the conical structure of the '839 appliance requires the use of a conical burr to prepare an artificial excavation at the fusion site prior to installation of the appliance. Use of a conical burr means the surgeon must remove a substantial amount of bone to create a space for implantation of the appliance. However, surgeons believe excess bone removal may be undesirable at the fusion site. In addition, the screws that are utilized to encourage compression of the carpal bones together are loaded largely in tension because of the angle in which they enter the bones of the carpus which may increase the risk of screw loosening and subsequent failure of the procedure.

Bone graft is often placed into the joint space at the fusion site prior to attaching an appliance to the bones for fusion but it is desirable to place bone graft into the joint space after the appliance is attached or partially attached because the appliance will prevent displacement of the bones during impaction grafting and because compression of bones and the graft encourages bone growth and fusion.

However, current appliances tend to interfere with the packing of bone graft into the midcarpal joint after fusion. Although the '839 appliance includes a small round central hole, placing bone graft into the midcarpal joint during four corner fusion can be difficult because the appliance interferes with access to the joint space. Thus, the surgical arts would benefit from an appliance to assist in performing four corner fusion of the bones of the carpus that would require minimal resection of the carpal bones and that would allow the application of bone fasteners preferably not loaded in tension. It would also be beneficial if the appliance allowed for ready placement of bone graft material within the joints between the bones of the carpus after the appliance is in place. Further, it would be valuable to have a bone burr specifically designed for four corner fusion of the capitate, hamate, lunate, and triquetrum bones that allowed implantation of a fusion appliance. It would also be helpful if the appliance were usable in patients suffering from osteoarthritis, posttraumatic arthritis, fractures, revision of failed partial wrist fusion, carpal instability and rheumatoid arthritis.

### SUMMARY OF THE INVENTION

The present invention solves many of the above-discussed problems by providing a low profile carpal compression plate utilized along with bone screws of an aggressive screw design. The screws are loaded largely in shear when the plate is in place. The present invention requires minimal bone resection, which may be performed with the assistance of a bone burr that forms part of one embodiment of the invention.

The low profile of the carpal compression plate of the invention requires minimal bone resection. Less bone resection allows for more accurate screw placement and greater bone purchase for each screw placed.

The fusion plate of the present invention includes at least one slotted aperture therein that is elongate in structure and which is narrower at a peripheral end and countersunk or otherwise shaped to be broader at a central end. Thus, the fusion plate of the present invention facilitates reduction and compression of the midcarpal bones and maintains effective fixation. Compression of the carpus is improved because one or more the screws are tightened into the slotted apertures they tend to migrate from the more peripheral portion of the slotted aperture to the more central portion of the slotted aperture, thus drawing the bones attached to these screws to be fused tightly together.

The fusion plate of the present invention further includes a multi-lobate central aperture to provide ease of access to the joint space between the capitate, hamate, lunate, and triquetrum bones after fixation of the fusion plate. This access allows easier placement of bone graft and permits secondary manipulation, for example, for the correction of DISI deformities (dorsal intercalated segment instability).

The present invention also includes a surgical bone fusion kit that incorporates bone fusion plates, K-wires, bone screws, a burr, a depth gauge and a burr handle into a single use disposable unit.

The present invention also includes a method of fusing bones of a carpus of a hand. The method includes surgically accessing the carpus and then rasping a concave depression in the carpus. Once the carpus is prepared the fusion plate is placed into the depression. Bone screws are inserted into the round screw hole and the elongate slotted apertures into bones of the carpus and tightened to compress the bones together.

The bone screws of the present invention are self-tapping and have an aggressive thread profile that improves retention of the screws in the bone and aids in the effective compression of bones together to facilitate bone fusion.

### BRIEF DESCRIPTION OF THE DRAWING

FIG. 1 is a perspective view of a fusion plate in accordance with an aspect of the present invention;

FIG. 2 is a top plan view of the fusion plate;

FIG. 3 is an elevational view of the fusion plate of FIG. 1;

FIG. 4 is a perspective view of a fusion plate, including bone screws in accordance with the present invention;

FIG. 5 is a perspective partially phantom view of a fusion plate and bone screws in situ in a human carpus;

FIG. 6 is an additional plan view of a fusion plate;

FIG. 7 is a plan view of the fusion plate, including section lines;

FIG. 8 is a cross sectional view of the fusion plate of FIG. 7 taken along section line 8-8;

FIG. 9 is a cross sectional view of the fusion plate of FIG. 7 taken along section line 9-9;

FIG. 10 is a plan view of the fusion plate showing a draft at the periphery thereof;

FIG. 11 is a top perspective view of the fusion plate;

FIG. 12 is a bottom perspective view of the fusion plate;

FIG. 13 is a perspective view of a bone burr in accordance with the present invention;

FIG. 14 is a plan view of a bone burr in accordance with the present invention;

FIG. 15 is an alternative plan view of the bone burr in accordance with the present invention showing the design of spiral teeth;

FIG. 16 is an elevational view of the bone burr;

FIG. 17 is a perspective view of a bone screw in accordance with the present invention;

FIG. 18 is an elevational view of the bone screw in accordance with the present invention;

FIG. 19 is a cross sectional of the bone screw of FIG. 18 taken along section line 19-19;

FIG. 19a is a detailed view of the thread structure of the bone screw of FIG. 19;

FIG. 19b is a detailed view of the head of the bone screw of FIG. 19;

FIG. 20 is a graph of screw pull-out strength from testing of bone screws in accordance with the present invention;

FIG. 21 is a perspective view of a bone fusion kit in accordance with the present invention;

FIG. 22 is an additional perspective view of a bone fusion kit of FIG. 21;

FIG. 23 is a perspective view of a bone fusion kit in accordance with the present invention with fusion plates removed and a burr cover removed;

FIG. 24 is a perspective view of a bone fusion kit with a K-wire dispenser advanced;

FIG. 25 is a perspective view of a bone fusion kit with a depth gage partially advanced;

FIG. 26 is a perspective view of a bone fusion kit showing the operation of a screw dispenser; and

FIG. 27 is an exploded perspective view of a bone fusion kit.

### DETAILED DESCRIPTION OF THE INVENTION

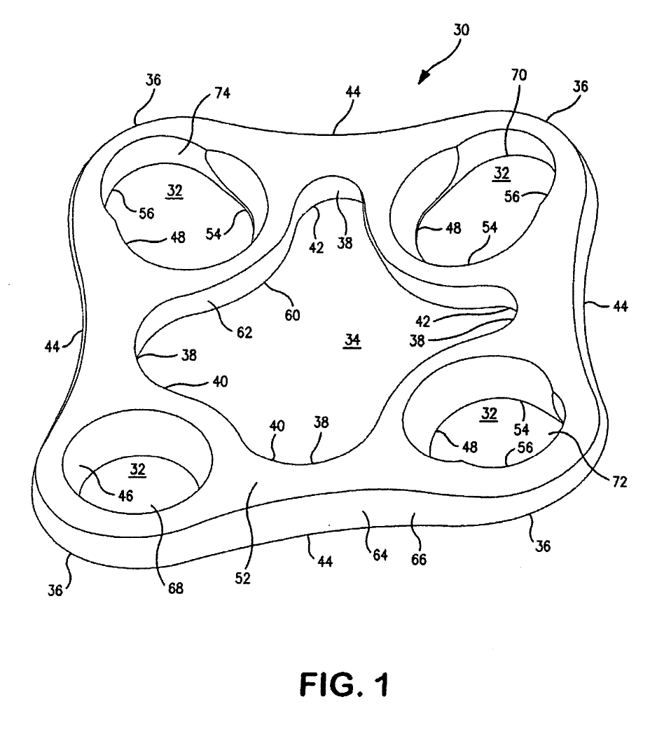

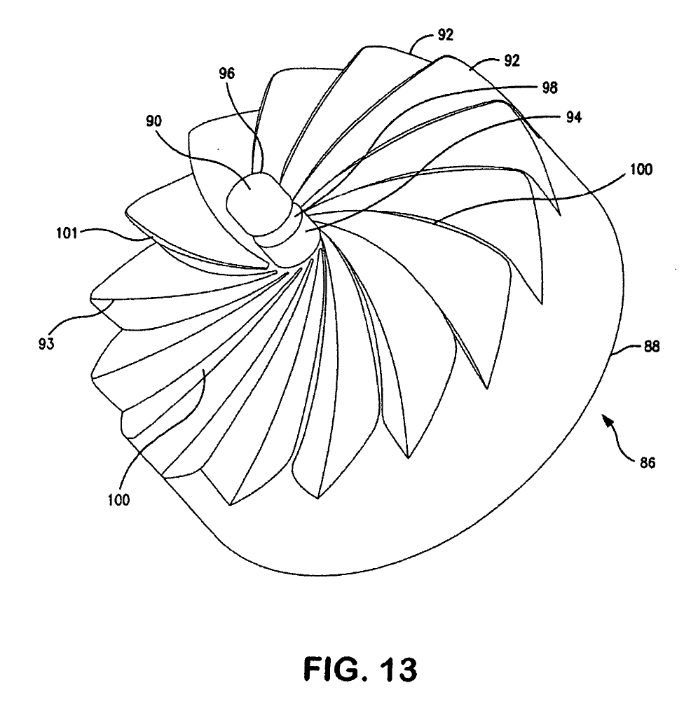

The carpal fusion plate 30 of the present invention is concave in profile to facilitate low profile implantation. Referring to FIGS. 1-3 and 6-12, the carpal fusion plate 30 in one embodiment may be generally diamond shaped and defines screw holes 32 and central opening 34. Screw holes 32 may be located generally at four corners 36 of fusion plate 30. Other shapes may be utilized in forming fusion plate 34 including but not limited to circular.

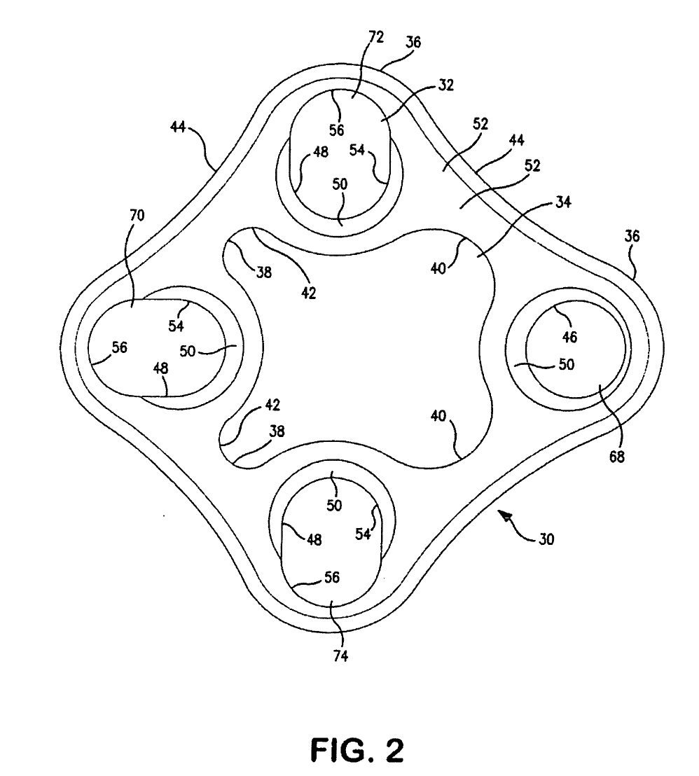

Referring to FIG. 2, central opening 34 may be multilobated and generally has four sides. Central opening 34 generally has four corners 38. Corners 38 of central opening 34 are generally located about midway between corners 36 of fusion plate 30. Central opening 34 includes two major lobes 40 and two minor lobes 42. The two major lobes 40 and two minor lobes 42 are located at adjacent corners.

Fusion plate 30 may be manufactured from implant grade 316L Stainless Steel. Other biocompatible materials such as titanium may also be utilized. Fusion plate 30 may be mirror polished over at least the top face 52 of fusion plate 30 or over its entire surface to facilitate soft tissue and tendon excursion. Alternately, bottom face 43 of fusion plate may be roughened to encourage osseointegration such as by commercially pure titanium plasma coating. Fusion plate 30 may vary in size depending upon its application. For example, two sizes may be made available to accommodate variations in patient anatomy. For example, for use in fusing the human carpus a small fusion plate 30 may have dimensions of approximately 18.0 mm by 17.5 mm. A large fusion plate 30 may have dimensions of approximately 20.5 mm by 20.0 mm. These sizes may also be appropriate for other procedures such as tarsal-metatarsal fusion. Other sizes may be utilized, as necessary, for the fusion of other bones or bone fragments. These dimensions are exemplary only and should not be considered to be limiting.

Corners 36 at the periphery of fusion plate 30, when present, are smoothly rounded. Peripheral sides 44 of fusion plate 30 present a somewhat concave contour between corners 36.

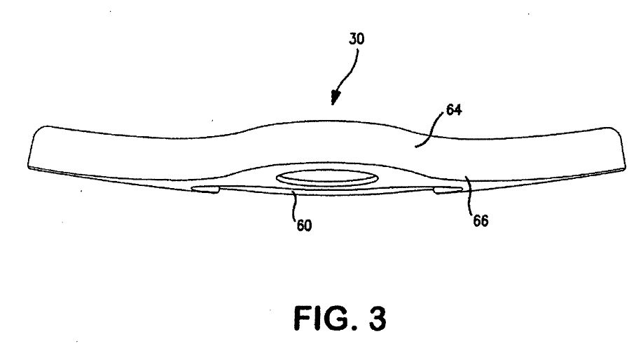

Referring particularly to FIGS. 1 and 3, fusion plate 30 has a generally spherical concave curvature. For example, fusion plate 30 may have a radius of curvature of from about one to about three inches.

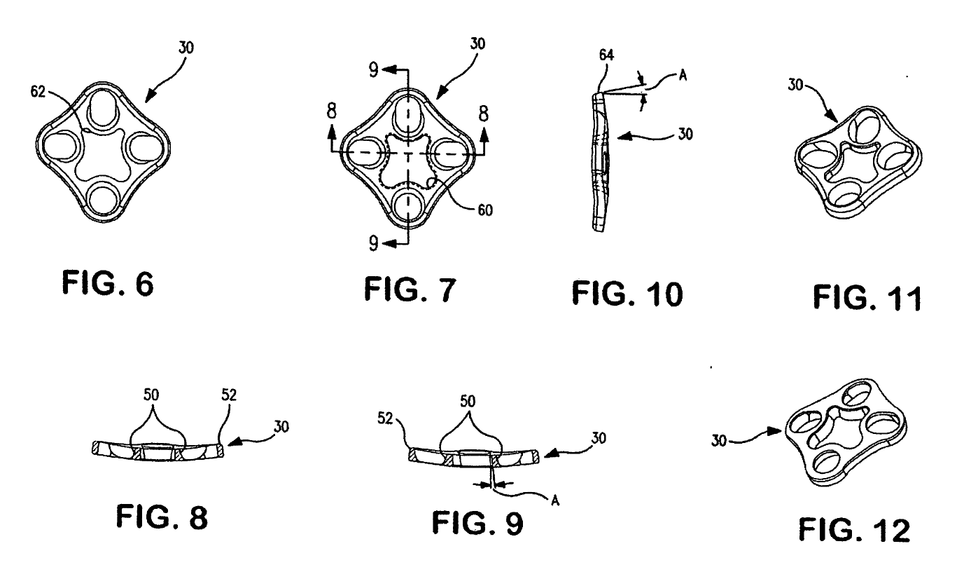

In one embodiment of the invention, screw holes 32 are located generally at corners 36 of fusion plate 30. At least one of screw holes 32 is a round screw hole 46 and at least one of screw holes 32 is a slotted aperture 48. Round screw hole 46 is countersunk, optionally with a spherical countersink 50. Referring to FIGS. 6-12 and particularly FIGS. 8-9, spherical countersink 50 is preferably positioned so that its center of curvature is located approximately at the top face 52 of fusion plate 30.

Slotted aperture 48 is tapered to include a broader portion 54 and a narrower portion 56. Broader portion 54 is located more centrally relative to fusion plate 30 and narrower portion 56 is located more peripherally relative to fusion plate 30. Slotted aperture 48 may be tapered throughout the entire thickness of fusion plate 30 or may be shaped such that it is broader only at one surface of the fusion plate 30. For example, slotted aperture 48 may be oval or racetrack shaped and may be countersunk at one end so that only the portion adjacent to one face of the fusion plate 30 is broader centrally than peripherally. In another embodiment of the invention, slotted aperture 48 includes an inclined plane that is thicker peripherally and thinner centrally.

Broader portion 54 of slotted aperture 48 may include a spherical countersink 50. Referring particularly to FIG. 8, the center of curvature of spherical countersink 50 desirably is located about even with top face 52 of fusion plate 30. Desirably fusion plate 30 includes one round screw hole 46 and three slotted apertures 48, though other numbers of round screw holes 46 and slotted apertures 48 may be utilized.

As best seen in FIGS. 3, 4, 7 and 9, central opening 34 desirably includes draft 60 around the top face of periphery 62 thereof. Draft 60 is formed at an angle A of about ten degrees. Fusion plate 30 also includes a draft 64 about the top face of periphery 66 thereof. Draft 64 also desirably forms an angle B equaling about ten degrees. Draft 64 is positioned so that draft 64 reduces the size of top face 52. Draft 60 is angled to reduce the size of bottom face 43.

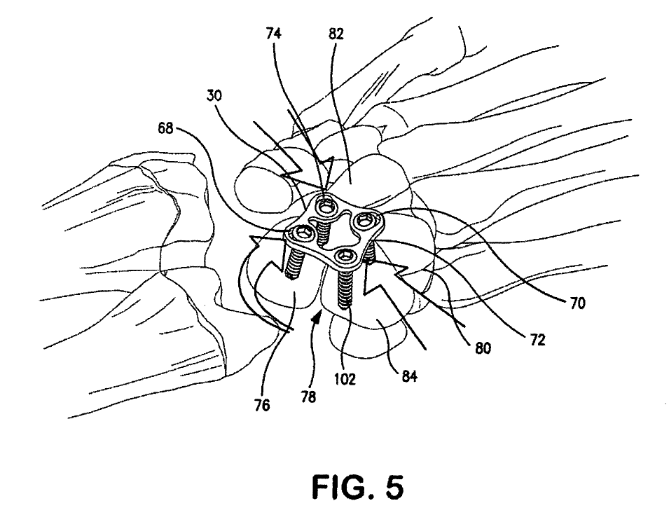

Screw holes 32 may also be defined as including lunate screw hole 68, hamate screw hole 70, right screw hole 72, and left screw hole 74. Generally, round screw hole 46 coincides with lunate screw hole 68. When fusion plate 30 is viewed so that round screw hole 46, which generally coincides with lunate screw hole 68 is at the bottom, hamate screw hole 70 is at the top. Referring to FIG. 5, when lunate screw hole 68 is aligned with the lunate 76 of a human carpus 78, hamate screw hole 70 will align with the hamate 80. Right screw hole 72 will align with the capitate 82 for the left hand and with the triquetrum 84 for the right hand. Left screw hole 74 will align with the triquetrum 84 for the left hand and with the capitate 82 for the right hand. In one embodiment, at least one of or all of lunate screw hole 68, hamate screw hole 70, right screw hole 72, and left screw hole 74 may be slotted apertures 48.

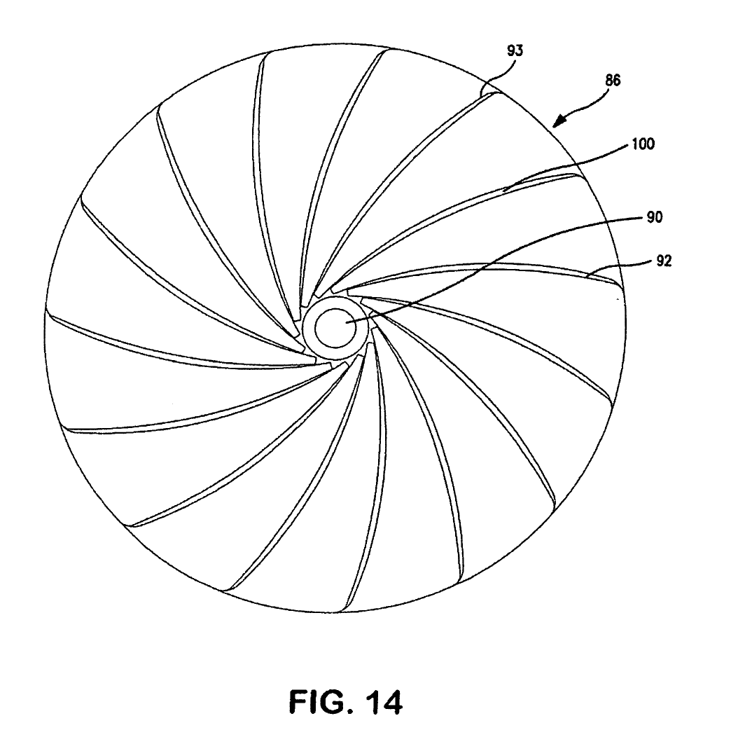

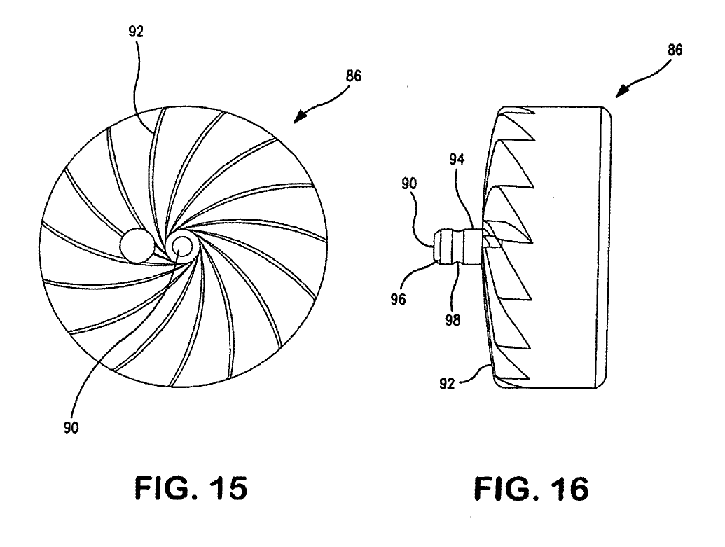

Referring to FIGS. 13-16, the invention also includes a burr 86 for preparing the carpus for implantation of the fusion plate 30. Burr 86 generally includes burr body 88, pilot stem 90 and spiral teeth 92. Burr body 88 is generally cylindrical in structure and may be adapted to be connected to a handle or driver or to a bit that can be secured into a power driver, if desired. Preferably, burr 86 is secured to a handle (not shown) for manual operation. Pilot stem 90 protrudes from the center of burr body 88. Pilot stem 90 generally includes stem 94 with rounded end 96, and groove 98. Groove 98 is situated generally around circumference of stem 94.

Burr 86 includes spiral teeth 92. Desirably burr 86 includes fifteen spiral teeth 92 at twenty-four degree increments. Spiral teeth 92 desirably have a sixty-degree helical tooth pattern, that is spiral teeth 92 are cut so that the angle formed at the cutting edge 93 is about sixty degrees. Spiral teeth 92 are machined so that spiral teeth begin their trajectory at a start point substantially tangent to the circumference of pilot stem 90.

Spiral teeth 92 desirably have a flat 100 along edge 101. Flat 100 desirably begins at the outer periphery of spiral teeth 92 and gradually decreases until dead sharp at the pilot stem 90 of burr 86.

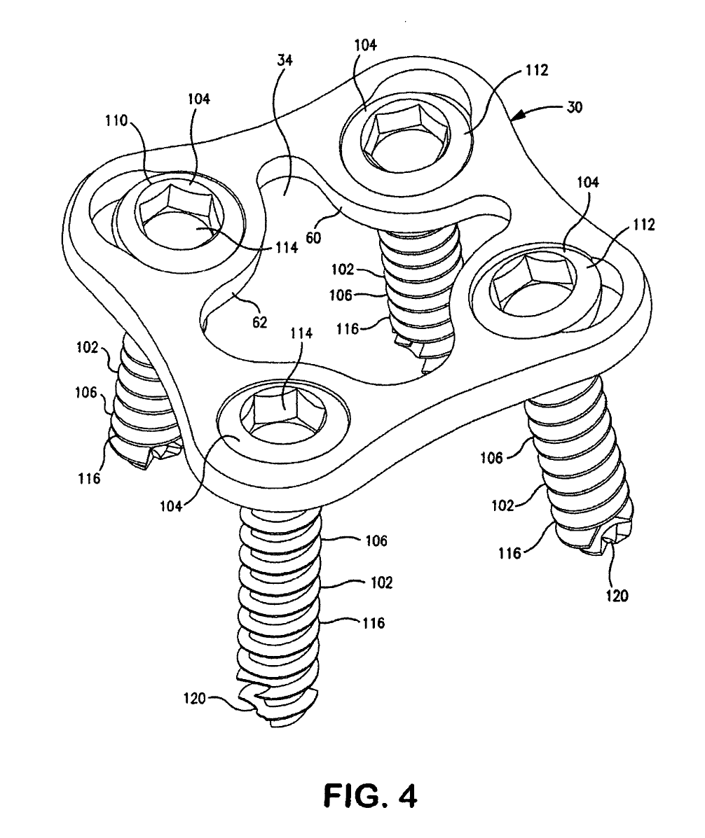

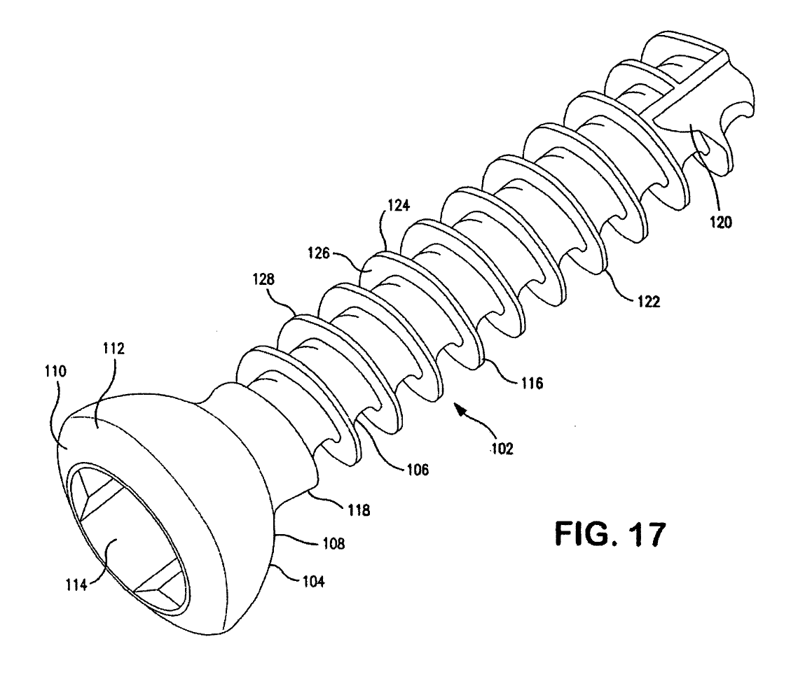

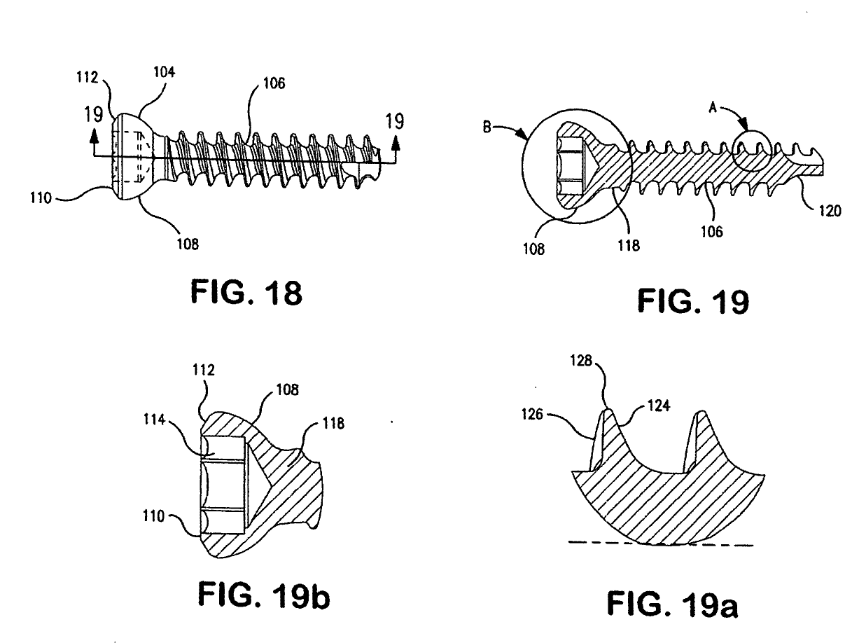

Referring to FIGS. 4-5 and 17-21, the invention further includes bone screws 102. Bone screws 102 generally include head 104 and shaft 106.
Referring particularly to FIGS. 17, 18, 19 and 21, head 104 is a spherical low profile head. Head 104 generally includes spherical portion 108, flattened face 110 and bevel 112, where spherical portion 108 meets flattened face 110. Bevel 112 may alternately be formed as a radius. Head 104 also includes screw interface 114 that is desirably adapted to receive a 2.5 mm hexagonal screwdriver, but can be formed to interface with any screwdriver known in the art. Carpal screws 38 may be manufactured to the standards of ISO 5835.

Shaft 106 generally includes threaded portion 116 and unthreaded portion 118. Unthreaded portion 118 desirably includes a taper that is narrower toward head 104 and broader toward threaded portion 116. Threaded portion 116 includes at least one flute 120 to make bone screw 102 self-tapping. Shaft 106 may be somewhat smaller in diameter than that of conventional bone screws.

Threaded portion 116 includes threads 122. As best seen in FIG. 19a, threads 122 include distal face 124 on a side distal to head 104, and proximal face 126. As can be seen in FIG. 19a, distal face 124 is angled more steeply than proximal face 126. Desirably distal face 124 is angled about twenty-five degrees to a normal from shaft 106, and proximal face 126 is angled about three degrees. Threads 122 also define radiused edge 128. If shaft 106 is smaller in diameter than conventional bone screws as discussed above threads 122 may be deeper thus increasing holding power of bone screws 102.

Desirably bone screws 102 are formed from 316L Stainless Steel or another rigid biocompatible material of appropriate strength known to the orthopedic arts such as titanium.

In operation, fusion plate 30 is implanted overlying the lunate 76, hamate 80, capitate 82 and triquetrum 84 bones of a human carpus 78. These midcarpal bones define a midcarpal joint. Once the carpus 78 is exposed, burr 86 is utilized to rasp the midcarpal bones. Pilot stem 90 of burr 86 is inserted into the juncture of the lunate 76, capitate 82, hamate 80 and triquetrum 84 bones or another group of bones to be fused, and using medium manual pressure, burr 86 is rotated to create a concave depression on the dorsal aspect of the lunate 76, hamate 80, capitate 82 and triquetrum 84 bones.

An appropriately sized fusion plate 30 is placed so that lunate screw hole 68, which is also round screw hole 46, overlays the lunate 76. A pilot hole is drilled and bone screw 102 is inserted through lunate screw hole 68. Hamate screw hole 70 is positioned over the hamate 80, and right screw hole 72 and left screw hole 74 are positioned over the capitate 82 and triquetrum 84. Pilot holes are drilled at the peripheral end of slotted apertures 48 and bone screws 102 are inserted but not tightened into the pilot holes. Bone graft is then added into the midcarpal joint and bone screws 102 are tightened. Referring to FIGS. 4 and 5, as bone screws 102 are tightened into slotted apertures 48, heads of bone screws 104 are drawn toward the center of fusion plate 30 by the tapered nature of slotted apertures 48 and spherical countersink 50 of slotted aperture 48, thus drawing together the lunate 76, hamate 80, capitate 82 and triquetrum 84 bones. The compression provided by the tightening of bone screws 102 into fusion plate 30 facilitates fusion of the bones of the carpus 78 and bone graft. The range of hand motion is assessed and the capsule and skin are closed.

Note that because of the orientation of screws 102 and the configuration of slotted apertures 48, when tightened, screws 102 are loaded primarily in shear thereby reducing the risk of screw loosening as opposed to screws that are inserted into bone and loaded primarily in tension.

The foregoing provides an overview of the surgical implantation procedure. A detailed description of the surgical procedure can be found below.

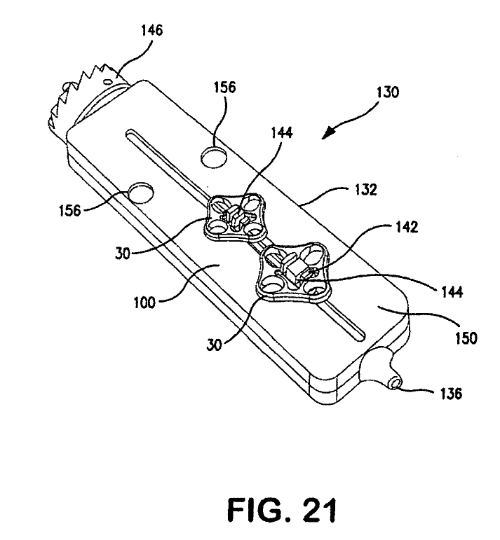

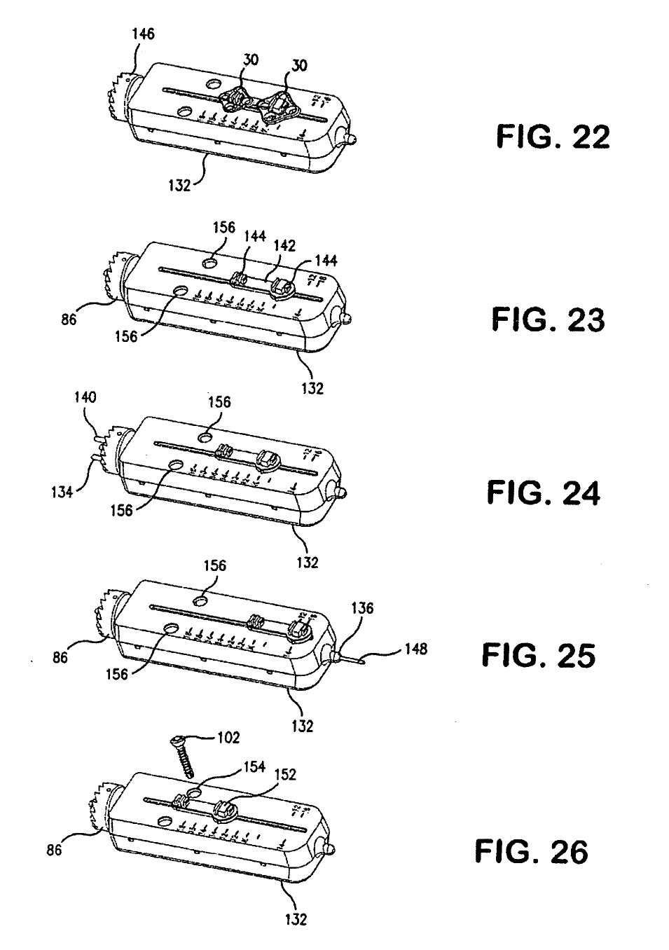

In one embodiment of the invention one or more of fusion plates 30 is supplied as art of a surgical bone fusion kit 130 depicted in FIGS. 21-26. Bone fusion kit 130 includes one or more fusion plates 30, burr 86, bone screws 102, housing 132, K-wire dispenser 134, depth gauge 136, screw selector/dispenser 138; K-wires 140, and slider 142. Referring to FIGS. 21 and 22, fusion plates 30 of various sizes are secured to slider 142. Fusion plates 30 may be secured to slider by an interference snap fitting 144. Fusion plates 30 may be removed from snap fitting 144 by lifting to overcome the interference tension of snap fitting 144.

Housing 132 is desirably an oblong structure appropriately sized to fit in the hand and also to act as a handle for burr 86. Bone fusion kit 130 may also include a burr cover 146. Burr cover 146 may conveniently be formed to snap fit over pilot stem 90 to secure it in place.

Slider 142 serves multiple purposes. As seen in FIG. 24, when slid into an end of housing 132 near burr 86, slider 142 advances K-wires 140 from K-wire dispenser 134 which may pass K-wires through an aperture in burr 86. When K-wire dispenser 134 is advanced, K-wires 140 may be grasped by hand or with a surgical tool for removal from housing 132. Referring to FIG. 23, K-wire dispenser 134 can be seen in advanced position so that K-wires 140 are exposed through burr 86.

Referring to FIG. 25, slider 142 further is operably attached to depth probe 148. As slider 142 is advanced away from burr 86, it advances depth probe 148 from the end of housing 132. Housing 132 may support indicia 150 so that pointer 152 points at appropriate indicia 150 indicating the depth of a hole probed by depth probe 148. Referring to FIG. 24, depth probe 148 can be seen in an advanced position.

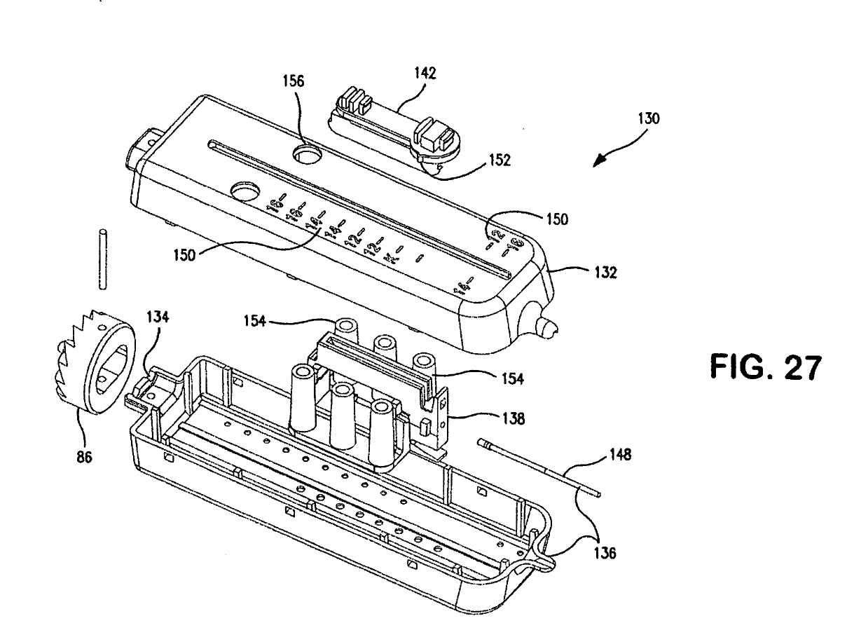

Referring to FIGS. 26 and 27, slider 142 in combination with housing 132 also actuates screw selector/dispenser 138. Slider 142 is interconnected with screw storage compartments 154. Multiple screw storage compartments 154 are slideably arranged within housing 132 and operably connected to slider 142. Additional indicia 150 located on housing 132 indicate the stored sizes of bone screws 102 stored within housing 132. Indicia 150 may be arranged so that pointer 152 indicates an appropriate indicia 150 indicating the length of bone screws 102 in one of screw storage compartment 154, which will be aligned with screw dispensing port 156. When slider 142 is manipulated so that pointer 152 points at an appropriate length indicia 150 housing 132 may be turned over to dispense a bone screw 102. Bone fusion kit 130 may be disposable in nature, so that after a single use of fusion plates 30 and bone screws 102, the rest of bone fusion kit 130 may be discarded. Thus bone fusion kit 130 provides convenient access to burr 86, to rasp a shallow depression for the placement of fusion plate 30. Bone fusion kit 130 also provides a readily available depth gauge 136 to determine the desired length for bone screw 102 once screw holes are formed in the bone. In addition, bone fusion kit 130 provides K-wires 140 conveniently available for use to drill holes and to provide fixation of bones as needed during the surgery. Burr cover 146 both protects burr 86 from harm and protects persons handling the bone fusion kit 130 from possible injury on the sharp spiral teeth 92 of burr 86.

Finally, bone fusion kit 130 keeps bone screws 102 conveniently available, sorted by size and readily dispensable at the time desired by the surgeon. Thus, bone screws are easily accessed and loss or misplacement is prevented. Only bone screws 102 that are needed for a surgical procedure need be dispensed thus reducing the risk of an excess screw accidentally being left in a body cavity.

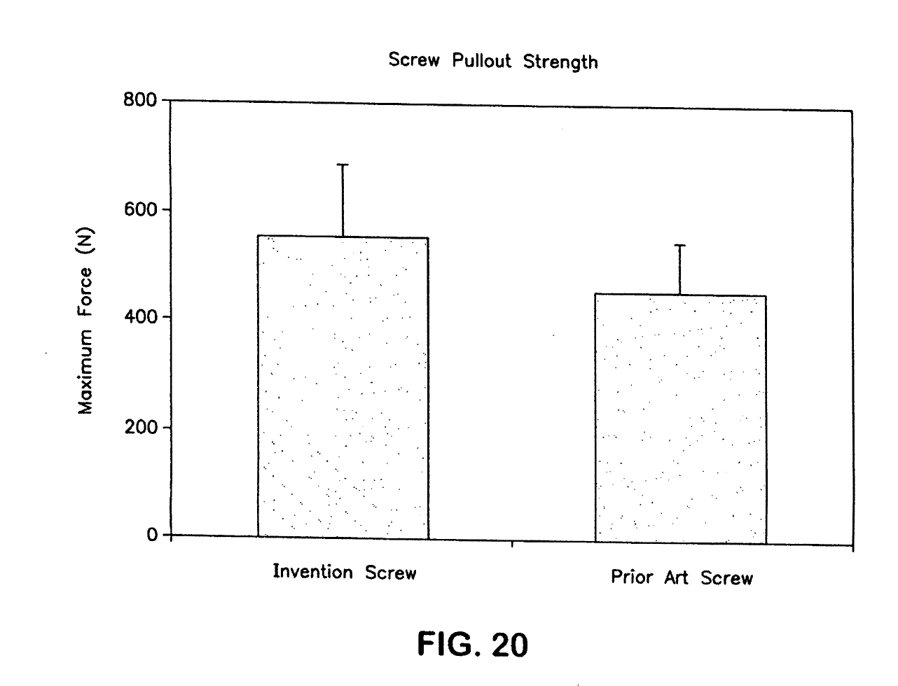

Screw pullout testing was performed to compare performance of bone screws 102 that may be used in accordance with the present invention with prior art bone screws. Researchers obtained two human cadaver radiuses from the same individual. Researchers placed three screws in accordance with the invention and three prior art screws in each radius specimen. The prior art screws were all of the same type. The screws were alternately placed and the order of the screws was reversed in the second specimen as compared to the first specimen. Screws were inserted through the dorsal cortex of the bone only and screw positioning was confirmed radiographically. The bone specimens were then placed in a holder and the screws were pulled out at 0.1 millimeters per second. Researchers recorded the maximum force achieved as the screws were pulled out. Test results are shown in the tables below and in a graph depicted in FIG. 20.

Results
Summary

| Invention vs. Prior Art | | | Invention/Prior Art each location | | | |
|---|---|---|---|---|---|---|
| | | Prior | | | Prior | |
| Trial | Invention | Art | Location | Invention | Art | Ratio |
| 1 | 715.6 | 577.0 | 1 | 715.6 | 419.9 | 1.70 |
| 2 | 559.0 | 510.9 | 2 | 707.3 | 577.0 | 1.23 |
| 3 | 497.4 | 438.9 | 3 | 559.0 | 468.0 | 1.19 |
| 4 | 707.3 | 419.9 | 4 | 443.8 | 510.9 | 0.87 |
| 5 | 443.8 | 468.0 | 5 | 497.4 | 312.3 | 1.59 |
| 6 | 395.0 | 312.3 | 6 | 395.0 | 438.9 | 0.90 |
| Average | 553.0 | 454.5 | | | Average | 1.25 |
| Std Dev | 134.4 | 89.5 | | | Std Dev | 0.35 |
| T-test | | | | | 95% Cl | 0.362 |

| | |
|---|---|
| p = 0.1658 | Cl [0.89, 1.61] |

| Specimen | Location* | Screw | Max Force (N) |
|---|---|---|---|
| 1 | 1 | Invention | 715.6 |
| 1 | 2 | Prior Art | 577.0 |
| 1 | 3 | Invention | 559.0 |
| 1 | 4 | Prior Art | 510.9 |
| 1 | 5 | Invention | 497.4 |
| 1 | 6 | Prior Art | 438.9 |
| 2 | 1 | Prior Art | 419.9 |
| 2 | 2 | Invention | 707.3 |
| 2 | 3 | Prior Art | 468.0 |
| 2 | 4 | Invention | 443.8 |
| 2 | 5 | Prior Art | 312.3 |
| 2 | 6 | Invention | 395.0 |

*Note:
Location 1 is most proximal, 6 is most distal

### Surgical Procedure

The following surgical procedure describes the bone fusion plate 30 of the invention as being utilized for “four corner” fusion of the capitate 82, hamate 80, lunate 76, and triquetrum 84 bones in the hand. This is exemplary and not intended to be limiting. The fusion plate 30 may be utilized for other fusion procedures involving other bones such as tarsal-metatarsal fusion in the foot.

Preoperative Assessment & Anatomy

Limited wrist fusion is often required in the treatment of advanced arthritis of the wrist. The most common conditions precipitating limited wrist fusion are scapholunate disassociation advanced collapse (SLAC wrist), and scaphoid non-union arthritic advanced collapse (SNAC wrist). Limited wrist fusion may also be required to treat failed treatment of perilunate fracture-dislocations of the wrist.

Indications

The fusion plate 30 is designed for “four-corner” fusion of the capitate 82, hamate 80, lunate 76, and triquetrum 84 bones in the hand and the fusion of other bones as desired by the surgeon. The fusion plate 30 is intended for use in patients suffering from pain and/or loss of function of the wrist due to osteoarthritis, post-traumatic arthritis, fractures, revision of failed partial wrist fusions, carpal instability, or rheumatoid arthritis. The fusion plate 30 may be made available in several different sizes to accommodate variations in patient anatomy and size. An appropriately sized fusion plate 30 will position over the capitate 82, hamate 80, lunate 76, and triquetrum 84 bones while not impinging on the dorsal rim of the distal end of the radius upon dorsiflexion of the hand when implanted.

Contra-Indications

The fusion plate 30 is contra-indicated if there are arthritic changes involving the radiocarpal joint. In cases of radio-scapho-luno fusions, the fusion plate 30 is contraindicated if the midcarpal joint (specifically the capitolunate or scaphocapitate joints) is arthritic.

Contraindications also include previous infection (sepsis) and may include failed silicone implants if advanced silicone synovitis would favor a complete wrist fusion or proximal carpal row resection.

Dorsal Incision

Open the skin with a dorsal midline or lazy “S” shaped incision on the distal radius centered on Lister's tubercle. A dorsal, “T” shaped incision may also be used. An inflatable tourniquet is typically used proximal to the wrist to control bleeding. A lazy “S” incision includes an incision that resembles a letter “S” or a reversed letter “S.”

Extensor Retinaculum Reflection

Make an incision in the extensor retinaculum between the first and second extensor compartments and reflect the extensor retinaculum ulnarly. This approach allows for later use of the extensor retinaculum for capsular repair, should it be desirable or necessary.

Capsular Exposure

Expose the capsule, with an “H” shaped distal flap, or a radial-based (Mayo) triangular flap.

Carpal Bone Exposure

Expose the carpal bones with distal wrist traction and soft tissue capsule retraction. Resect the proximal third to half of the scaphoid in the case of SLAC or SNAC Wrist. If desired a K-wire can be temporarily inserted into the scaphoid to assist in bone resection.

Midcarpal Joint Cartilage Removal

Remove cartilage from the midcarpal joint to create bone-to-bone contact to facilitate fusion of adjacent bones. Cartilage removal may be accomplished using a rongeur, osteotome, or curette. Occasionally for hard bone, a bone burr may be needed. Cancellous autograft may be obtained from the distal radius (elevating Lister's tubercle), proximal ulna, or iliac crest. Bone graft material composed of cancellous allograft is now placed volar to dorsal between the bones at the midcarpal fusion site.

Reduction of Lunate

Identify and reduce the lunate in alignment with the capitate using a K-wire.

Reduction of Midcarpal Joints

Use additional K-wires to hold the midcarpal joint in alignment. This includes using a K-wire to maintain reduction of the lunate.

Rasping of Midcarpals

Use burr 86 to resect cortical bone from the dorsal surfaces of the lunate 76, capitate 82, hamate 80, and triquetrum 84. Cortical bone resection is desired in order to properly seat the fusion plate 30. Use of a manual burr in this step is recommended instead of a power driven burr. Manual rasping of the midcarpals significantly reduces the likelihood of bone necrosis caused by heat generation. A smooth, contoured surface conforming to the shape of the fusion plate 30 should be created. Any additional bone fragments can be removed using a rongeur.

Plate Selection and Placement

Determine the appropriate size plate. Center the selected fusion plate 30 over the four-corner bone fusion site. The round screw hole 46 should be aligned with the lunate 76. Align the slotted apertures 48 over the hamate 80, capitate 82 and triquetrum 84. Temporarily hold the plate in position while dorsi-flexion is performed to assess whether or not there will be any impingement of the fusion plate 30 with the dorsal ridge of the radius.

The round screw hole 46 should be located over the lunate 76 approximately one millimeter distal to the articular cartilage of the lunate 76. Placement should be accurate to prevent the fusion plate 30 from impinging on the radius in dorsi-flexion. If impingement does occur, move the plate distally, use the burr 86 to remove additional bone to further recess the plate, and/or ascertain that an appropriately sized plate is being used. It is important to avoid contact between the fusion plate 30 and the dorsal ridge of the dorsi-flexed radius.

Lunate Pilot Hole and Screw Placement

With the carpal bones reduced and the plate properly aligned over the lunate, use a K-wire to prepare a pilot hole in the lunate. Insert a bone screw through the lunate screw hole 68 but do not tighten it at this time.

A depth gauge can be advanced into the pilot hole to determine proper screw length if desired. Screw length should be selected so that the distal end of the screw does not extend through the bone.

Remaining Pilot Holes and Screw Placement

Drill pilot holes for the three remaining slotted apertures 48. Drill the pilot holes with a K-wire. Position the pilot hole at the peripheral edge of each slotted aperture 48. The slotted apertures 48, with bone screws 102 started at their outer edge, will radially compress the carpal bones as the screws are tightened into slotted apertures 48. At this time drill the pilot holes and place the bone screw 102 loosely. Do not tighten the bone screw 102 yet. Place the bone screws 102 in the following sequence: hamate 80, triquetrum 84 then capitate 82 unless otherwise required by the patient's wrist morphology.

Bone Graft

Place additional bone graft obtained from Lister's tubercle, the previous resection of the scaphoid, or other options mentioned earlier into the midcarpal fusion site as needed. Bone graft may be placed through central opening 34. The shape of central opening 34 facilitates access to the midcarpal fusion site for bone graft placement and compaction.

Screw Tightening

After bone graft has been inserted, tighten bone screws 102 to radially compress the bones together. Tighten the lunate bone screw first then the hamate 80, triquetrum 84 and capitate 82 bone screws 102 in sequence. The central opening 34 of the fusion plate 30 also allows manipulation of the bone graft while reducing the bones. Remove the temporary K-wires.

Range of Motion Assessment

Using C-arm imaging, move the hand in all planes to determine the stability of the fusion. Look for impingement between the lunate and the rim of the dorsal radius in extension. If impingement is present, consider trimming back the dorsal edge of the radius to prevent the occurrence of plate-bone impingement, or consider additional options as discussed above.

Capsular Closure

Close the dorsal wrist capsule with 3-0 or 4-0 absorbable sutures. The extensor retinaculum may be used to provide additional soft tissue reinforcement for the dorsal capsule. The distal third of the extensor retinaculum previously preserved can be rotated beneath the extensor tendons to reinforce the dorsal capsule of the wrist to further reduce the risk of the extensor tendons abrading on the fusion plate 30.

Final Capsule Repair and Closure

Repair the remaining extensor retinaculum (proximal two-thirds) back to the first extensor compartment with a 3-0 or 4-0 resorbable suture. Deflate the tourniquet, obtain hemostasis and close the subcutaneous tissue.

Follow Up

Post-operative treatment includes a compressive dressing with support splints for 48 hours followed by cast immobilization for 6 weeks. Rigid internal fixation of the midcarpal fusion site may allow for cast removal in 4 weeks, and the use of removable splints with earlier range of motion rehabilitation. Biplanar X-rays (in some cases computed tomography) is used to confirm a solid intercarpal fusion. Anticipated range of motion is 30-40° extension, 25-30° flexion and total of 40° radio ulnar deviation, which matches the range of motion needed for most daily activities.

Technique Tips

Screw Placement

Depth gauge measurement and C-arm imaging are valuable aids in determining proper screw length. Caution must be used to prevent the triquetral screw from passing through the triquetrum 84 into the pisiform.

Plate Positioning

Temporary plate positioning over the dorsal aspect of the midcarpal joint is necessary to be certain that the plate does not impinge against the dorsal distal radius rim during dorsiflexion of the hand.

Bone Grafting

Bone grafting volar to dorsal between the midcarpal bones should be performed before temporary K-wire fixation. Dorsal impaction grafting is then performed after the fusion plate 30 is in place. After the impaction grafting, bone screws 102 are sequentially tightened as indicated above to achieve compression of the carpals.

The present invention may be embodied in other specific forms without departing from the central attributes thereof, therefore, the illustrated embodiment should be considered in all respects as illustrative and not restrictive, reference being made to the appended claims rather than the foregoing description to indicate the scope of the invention.

1-33. (canceled)

34. A method of fusing bones or portions of bones, the method comprising the steps of:
surgically accessing the bones to be fused;
rasping a shallow concave depression in the bones to be fused;
placing a shallow curved plate into the depression, the plate having a top face and a bottom face, the bottom face being convex, the plate defining at least one round screw hole therethrough and at least one elongate slotted aperture there through, the at least one elongate slotted aperture having a long axis and being oriented such that the long axis extends from peripherally to centrally relative to the plate and the slotted aperture further being shaped such that it receives the head of the bone screw more deeply therein at a central end thereof than at a peripheral end thereof thereby causing the bone screw t transit centrally as the head of the bone screw is more deeply received;
placing bone screws through the round screw hole and the at least one elongate slotted aperture into bones; and
tightening the bone screws to compress the bones together.

35. The method as claimed in claim 34, further comprising the step of locating the concave depression substantially at the juncture of the lunate, hamate, capitate and triquetrum bones of the carpus.

36. The method as claimed in claim 34, further comprising the step of locating the round hole over the lunate bone.

37. The method as claimed in claim 34, further comprising the steps of inserting bone graft into a joint space between the bones to be fused once prior to placement of the plate and again after the placement of the plate through an opening in the plate.

38. The method as claimed in claim 34, in which the elongate slotted aperture is shaped so that it is broader adjacent to at least a top face of the plate at an end located more centrally relative to the plate and narrower at an end located peripherally relative to the plate.

39. The method as claimed in claim 34, further comprising the step of placing bone graft into a midcarpal joint through an opening in the plate.

40. The method as claimed in claim 34, in which the bone fusion plate is substantially diamond shaped.

41-53. (canceled)
